SEQUENCE LISTING (4 sequences)

SEQ ID NO: 1 - protein, 129 aa, Artificial Sequence
  Synthetic construct (Variable region of human Ab H chain)
QVKLLESGGGLVKPGGSLRLSCSASGFSLTKYKMTWVRQAPGKGLEWVSSISSTSRDIDYADSVKGRFTISRDNAKNSLFLQMSSLRVDDTAVYYCTRDGWLWGWDVRSNYYYNALDVWGQGTTVTVSS

SEQ ID NO: 2 - protein, 108 aa, Artificial Sequence
  Synthetic construct (Variable region of human Ab L chain)
ELVMTQSPSSLSASVGDRVTITCRASQGIYNSIAWYQQKPGKAPKLLLYSTSTLLSGVPSRFSGSGSGTDYTLTITNLQPEDFATYYCQQYFVTPETFGQGTKVEIKR

SEQ ID NO: 3 - DNA, 25 bp, Artificial Sequence
  primer M3
ctgggaggagttgggggaggagatt

SEQ ID NO: 4 - DNA, 19 bp, Artificial Sequence
  primer POL8
aggatagaacctagcaggc

(19) United States
(12) Patent Application Publication
(10) Pub. No.: US 2010/0260712 A1
(43) Pub. Date: Oct. 14, 2010

(54) USE OF HUMAN ANTIBODY CAPABLE OF NEUTRALIZING HEPATITIS B VIRUS FOR THE PREVENTION OR TREATMENT OF HEPATITIS B VIRUS INFECTION

(75) Inventors: Se-Ho Kim, Yongin-si (KR); Kwang-Won Hong, Yongin-si (KR); Yong-Nam Shin, Yongin-si (KR); Yong-Won Shin, Yongin-si (KR); Ki-Hwan Chang, Yongin-si (KR); Kyung-Hwan Ryoo, Yongin-si (KR); Jin-Seol Choi, Yongin-si (KR); Pan-Kyung Kim, Yongin-si (KR); Ki-Tae Bong, Yongin-si (KR); Dong-Hyuck Seo, Yongin-si (KR); Sun-Jeong Oh, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: GREEN CROSS CORPORATION, Yongin-si, Gyeonggi-do (KR)

(21) Appl. No.: 12/744,427

(22) PCT Filed: Nov. 21, 2008
(86) PCT No.: PCT/KR08/06868

§ 371 Date: Jun. 9, 2010

(30) Foreign Application Priority Data
Nov. 30, 2007 (KR) 10-2007-0123739

(51) Int. Cl. A61K 39/395; C07K 16/00; A61K 38/21; A61P 31/12

(52) U.S. Cl. 424/85.4; 530/387.1; 424/149.1; 424/161.1

(57) ABSTRACT

The present invention provides a use of a human antibody against a hepatitis B virus (HBV) surface antigen (HBsAg). The antibody exhibits an excellent HBV neutralizing ability, and thus, is useful for the prevention or treatment of the HBV infection or a disease caused thereby.

### FIELD OF THE INVENTION

The present invention relates to a use of a HBV neutralizing human antibody for the prevention or treatment of HBV infection or a disease caused thereby.

### BACKGROUND OF THE INVENTION

Hepatitis B virus (HBV) causes hepatitis and a liver cancer. The WHO has revealed that about ⅓ of chronic HBV patients develop liver cirrhosis or liver cancer, and about one million people die from HBV-associated diseases every year.

The development of a vaccine against HBV has made it possible to prevent hepatitis B, but many people are still suffering from chronic hepatitis by HBV infection. Further, there is an increasing demand for liver transplantation, which requires the development of an effective antibody that can inhibit HBV infection during the liver transplantation.

A virus replication inhibitor, such as lamivudine, is widely used as a therapeutic drug for chronic hepatitis B, but it is not possible to treat chronic hepatitis B by using only the virus replication inhibitor. A combination of the virus replication inhibitor with an antibody having a different action mechanism from the virus replication inhibitor is expected to lead to an increased therapeutic efficacy for chronic hepatitis B. Such an antibody may include a hepatitis B antibody (Hepatitis B Immune Globulin; HBIG) purified from a blood having a high anti-HBV antibody titer. However, the currently available HBIG is not an ideal source of a therapeutic antibody due to its limited availability, low specificity and possible contamination of infectious agents.

Accordingly, the use of a recombinant antibody has been attempted to solve the above problems. It is possible to be free from plasma availability and also possible to supply a safe product stably. It has been found that the activity of such a recombinant antibody is 3,000 times or more higher than that of a conventional plasma-derived antibody, which can be used to prevent viral infection during liver transplantation and to treat chronic hepatitis B.

### SUMMARY OF THE INVENTION

Accordingly, it is an object of the present invention to provide a use of a HBV neutralizing human antibody for the prevention or treatment of HBV infection or a disease caused thereby.

It is another object of the present invention to provide a method of preventing or treating HBV infection or a disease caused thereby using a HBV neutralizing human antibody.

In accordance with an aspect of the present invention, there is provided a use of a human antibody comprising a heavy chain variable region (V_{H}) having an amino acid sequence of SEQ ID NO: 1 and a light chain variable region (V_{L}) having an amino acid sequence of SEQ ID NO: 2 for the prevention or treatment of HBV infection or a disease caused thereby in a mammal.

In accordance with another aspect of the present invention, there is provided a method of preventing or treating HBV infection or a disease caused thereby in a mammal, the method comprising administering thereto a human antibody comprising a heavy chain variable region (V_{H}) having an amino acid sequence of SEQ ID NO: 1 and a light chain variable region (V_{L}) having an amino acid sequence of SEQ ID NO: 2 in a therapeutically effective amount.

### BRIEF DESCRIPTION OF DRAWINGS

The above and other objects and features of the present invention will become apparent from the following description of the invention taken in conjunction with the following accompanying drawings, which respectively show:

FIG. 1: the changes in the amounts of HBV DNA, HBsAg and anti-HBs in the blood sample collected from the inventive antibody-untreated chimpanzee;

FIG. 2: the structural features of HBV genome and plasmid pHBV1.3-MBRI which was used for hydrodynamic animal model;

FIG. 3: the change in the blood HBsAg concentration after administering the inventive antibody and/or an HBV replication inhibitor to pHBV1.3-MBRI plasmid injected mice;

FIG. 4: the change in the blood antibody concentration after administering the inventive antibody and/or an HBV replication inhibitor to pHBV1.3-MBRI plasmid injected mice;

FIG. 5(a): an immunoprecipitation analysis result showing the degree of the binding of the inventive antibody to HBV in patient blood according to the amount of the inventive antibody used;

FIG. 5(b): an immunoprecipitation analysis result showing the change in the amount of HBV DNA in patient blood according to the amount of the inventive antibody used;

FIG. 5(c): a comparative immunoprecipitation analysis result showing the degree of the binding of the inventive antibody or a conventional antibody to HBV in patient blood;

FIG. 6(a): an immunohistochemistry staining result showing the binding of the inventive antibody to an HBV-infected human liver tissue; and

FIG. 6(b): an immunohistochemistry staining result showing the binding of a negative control antibody with the same isotype as the inventive antibody to a HBV-infected human liver tissue.

### DETAILED DESCRIPTION OF THE INVENTION

A human antibody used in the method of preventing or treating HBV infection or a disease caused thereby comprises a heavy chain variable region (V_{H}) having an amino acid sequence of SEQ ID NO: 1 and a light chain variable region (V_{L}) having an amino acid sequence of SEQ ID NO: 2.

The present inventors have proved the HBV neutralizing efficacy of the inventive antibody in an experiment using chimpanzees. Specifically, the chimpanzees infused with a mixture of HBV and the inventive antibody had not been infected by HBV for one year of follow up. Further, when the inventive antibody was administered to an HBV-infected chimpanzee, the amount of an HBV surface antigen (HBsAg) started to decrease, but increased with a decrease of the amount of the inventive antibody. Immunoprecipitation analysis has shown that the inventive antibody strongly binds to HBV in the blood sample of a patient, and immunohistochemistry staining analysis has shown that the inventive antibody also strongly binds to an HBV-infected human liver tissue.

The human antibody used in the method of the present invention is disclosed in Korean Patent No. 467706, and could be produced by the cell line HBAb-49 (KCLRF-BP-00054).

As described in Examples of the present invention, the antibody shows an excellent neutralizing activity against HBV in animal experiments. Therefore, the antibody can be useful for the prevention or treatment of the HBV infection, for example, HBV infection during liver transplantation, and diseases caused thereby, e.g., chronic hepatitis.

Accordingly, the present invention also provides a pharmaceutical composition for the prevention or treatment of HBV infection or a disease caused thereby comprising a human antibody consisting of a heavy chain variable region (V_{H}) having an amino acid sequence of SEQ ID NO: 1 and a light chain variable region (V_{L}) having an amino acid sequence of SEQ ID NO: 2 as an active ingredient with a pharmaceutically acceptable carrier.

The pharmaceutical formulation can be formulated into various pharmaceutical formulations in accordance with any of the conventional procedures. In preparing a pharmaceutical formulation, the antibody is preferably admixed or diluted with a carrier, or enclosed within a container type carrier. When the carrier is used as a diluent, it may be a solid, semi-solid or liquid material acting as a carrier, excipient or medium for the antibody. Thus, the formulation may be in the form of a tablet, pill, powder, sachet, elixir, suspension, emulsion, solution, syrup, aerosol, soft or hard gelatin capsule, sterile injectable solution, sterile powder or the like.

Examples of suitable carrier, excipient and diluent are lactose, dextrose, sucrose, sorbitol, mannitol, calcium silicate, cellulose, methylcellulose, microcrystalline cellulose, polyvinylpyrrolidon, water, methylhydroxybenzoate, propylhydroxybenzoate, talc, magnesium stearate and mineral oil. The pharmaceutical composition may additionally include fillers, anti-agglutinating agents, lubricants, wetting agents, flavoring agents, emulsifiers, presevatives and the like. The pharmaceutical composition may also provide quick, sustained or delayed release of the antibody after its administration to a mammal by employing any of the methods well known in the art.

The inventive antibody can be used together with an antiviral drug such as interferon, an anti-HBV monoclonal antibody, an anti-HBV polyclonal antibody, a nucleoside homologue, a DNA polymerase inhibitor, an siRNA drug, or a therapeutic vaccine.

The inventive antibody can be administered to mammals including human to prevent or treat HBV infection or diseases caused thereby. The dosage of the antibody may be adjusted in light of various relevant factors such as the condition of the subject to be treated, type and seriousness of illness, an administration rate, and the opinion of doctor. The inventive antibody can be administered parenterally in an effective amount ranging from about 0.001 to 10 mg/kg (body weight)/dose, preferably 0.005 to 1 mg/kg/dose in a single dose or in divided doses. In certain cases, an amount less than the above dosage may be more suitable. An amount greater than the above dosage may be used provided that it does not cause serious side effects, and such a great amount can be administered in divided doses per day.

The following Examples are intended to illustrate the present invention without limiting its scope.

### Example 1

### Evaluation for HBV Neutralizing Ability in Chimpanzee

In order to confirm that the human antibody of the present invention shows the HBV neutralizing ability in vivo, the following experiment was carried out.

HBV 100 CID50 (50% chimpanzee infectious doses) obtained from Hepatitis Research Foundation (New York, USA) was put into three tubes. 0.1 mg and 10 mg of the inventive antibody (Hepabig-Gene, Green Cross, Korea) were added to the two tubes, respectively, and no antibody was added to the other tube. PBS (phosphate buffered saline) was added to the tubes to a final volume of 3 ml, the mixture was incubated at 37° C. for 1 hour and then at 4° C. overnight, and freezed with a liquid nitrogen to prepare a test sample.

The test sample was intravenously administered to three chimpanzees (Hepatitis Research Foundation, New York, USA), which had not been infected with HBV previously. The dosages of the antibody to each chimpanzee are listed in Table 1.

TABLE 1

| | | | Age | Body Weight | Dosage of |
|---|---|---|---|---|---|
| | | Sex | (Years) | (kg) | Antibody |
| Control | Chimpanzee 1 | Male | 4 | 12.2 | — |
| Test 1 | Chimpanzee 2 | Male | 4 | 11.6 | 0.1 mg |
| Test 2 | Chimpanzee 3 | Female | 4 | 10.8 | 10 mg |

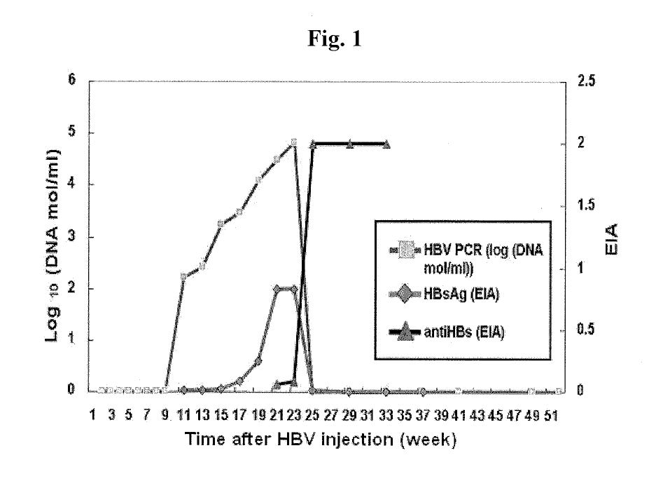

Blood samples were collected every one week from 1 week before the antibody administration to 8 weeks after the administration, and every 2 weeks from 8 weeks after the administration to measure markers related to the HBV infection such as an HBV DNA, HBsAg (HBV surface antigen), anti-HBs (antibody to HBsAg), anti-HBc (HBV core antibody), ALT and AST. Further, the in vivo safety of the antibody was evaluated through the blood and urine tests, and the results for the chimpanzees 1 to 3 are shown in Tables 2 to 4, respectively. Further, changes in the amounts of the HBV DNA, HBsAg and anti-HBs for the chimpanzee 1 were measured, and the results are shown in FIG. 1.

TABLE 2
(chimpanzee 1)

| | ALT | AST | HBV PCR Log10 | HBsAg | Anti-HBs | Anti-HBc |
|---|---|---|---|---|---|---|
| | (sf units) | (sf units) | (DNA mol/ml) | (EIA) | (EIA) | (EIA) |
| 1 week before | 6 | 11 | N | | | |
| Day of | 5 | 10 | N | | | |
| administration | | | | | | |
| 1 week after | 15 | 13 | N | | | |
| 2 weeks after | 6 | 16 | N | | | |
| 3 weeks after | 8 | 22 | N | | | |
| 4 weeks after | 2 | 6 | N | | | |
| 5 weeks after | 6 | 11 | N | | | |
| 6 weeks after | 5 | 12 | N | | | |
| 7 weeks after | 6 | 20 | N | | | |
| 8 weeks after | 7 | 18 | N | | | |
| 10 weeks after | 8 | 18 | 2.21 | .015(−) | | |
| 12 weeks after | 10 | 23 | 2.43 | .023(−) | | |
| 14 weeks after | 13 | 25 | 3.24 | .064(+) | | |
| 16 weeks after | 12 | 18 | 3.47 | .209(+) | | |
| 18 weeks after | 8 | 20 | 4.10 | .600(+) | | 1.004(−) |
| 20 weeks after | 8 | 13 | 4.50 | >2.000(+) | | 1.264(−) |
| 22 weeks after | 10 | 12 | 4.82 | >2.000(+) | .056(−) | 1.038(−) |
| 24 weeks after | 15 | 18 | N | .003(−) | .085(−) | .0129(+) |
| 28 weeks after | 23 | 22 | N | N | >2.000(+) | 0.156(+) |
| 32 weeks after | 19 | 18 | N | N | >2.000(+) | 0.119(+) |
| 36 weeks after | 21 | 19 | N | N | >2.000(+) | 0.061(+) |
| 40 weeks after | 7 | 23 | N | | | |
| 44 weeks after | 25, 24 | 19 | N | | | |
| 48 weeks after | 19 | 16 | N | | | |
| 51 weeks after | 28, 29 | 23 | N | | | |

TABLE 3
(chimpanzee 2)

| | | | HBV PCR | Anti- | Anti- |
|---|---|---|---|---|---|
| | ALT | AST | Log10 (DNA | HBs | HBc |
| | (sf units) | (sf units) | mol/ml) | (EIA) | (EIA) |
| 1 week before | 26 | 22 | N | | |
| Day of | 9 | 26, 25 | N | | |
| administration | | | | | |
| 1 week after | 10 | 23 | N | | |
| 2 weeks after | 6 | 24 | N | | |
| 3 weeks after | 9 | 25 | N | | |
| 4 weeks after | 4 | 18 | N | | |
| 5 weeks after | 9 | 37, 37 | N | | |
| 6 weeks after | 5 | 25 | N | | |
| 7 weeks after | 5 | 9 | N | | |
| 8 weeks after | 5 | 13 | N | | |
| 10 weeks after | 8 | 17 | N | | |
| 12 weeks after | 14 | 21 | N | | |
| 14 weeks after | 17 | 23 | N | | |
| 16 weeks after | 15 | 19 | N | | |
| 18 weeks after | 22 | 16 | N | | |
| 20 weeks after | 20 | 16 | N | | |
| 22 weeks after | 13 | 19 | N | | |
| 24 weeks after | 24 | 22 | N | | |
| 28 weeks after | 28, 28 | 25 | N | | |
| 32 weeks after | 24 | 26 | N | | |
| 36 weeks after | 23 | 25 | N | | |
| 40 weeks after | 11 | 20 | N | | |
| 44 weeks after | 27, 27 | 17 | N | | |
| 48 weeks after | 18 | 13 | N | N | |
| 51 weeks after | 30, 29 | 24 | N | | N |

TABLE 4
(chimpanzee 3)

| | ALT | AST | HBV PCR Log10 | HBeAg | Anti-HBe | Anti-HBs | Anti-HBc |
|---|---|---|---|---|---|---|---|
| | (sf units) | (sf units) | (DNA mol/ml) | (EIA) | (EIA) | (EIA) | (EIA) |
| 1 week before | 5 | 18 | N | | | | |
| Day of | 11 | 22 | N | | | | |
| administration | | | | | | | |
| 1 week after | 7 | 18 | N | | | | |
| 2 weeks after | 5 | 20 | N | | | | |
| 3 weeks after | 13 | 31, 31 | N | | | | |
| 4 weeks after | 9 | 19 | N | (−) | (−) | | |
| 5 weeks after | 8 | 23 | N | (−) | (−) | | |
| 6 weeks after | 14 | 26 | N | (−) | (−) | | |
| 7 weeks after | 7 | 15 | N | (−) | (−) | | |
| 8 weeks after | 10 | 19 | N | (−) | (−) | | |
| 10 weeks after | 20 | 16 | N | | | | |
| 12 weeks after | 13 | 19 | N | | | | |
| 14 weeks after | 16 | 21 | N | | | | |
| 16 weeks after | 16 | 24 | 2.24*, N, N | | | | |
| 18 weeks after | 21 | 24 | N | | | | |
| 20 weeks after | 14 | 22 | N | | | | |
| 22 weeks after | 16 | 19 | N | | | | |
| 24 weeks after | 21 | 17 | N | | | | |
| 28 weeks after | 18 | 21 | N | | | | |
| 32 weeks after | 23 | 16 | N | | | | |
| 36 weeks after | 22 | 17 | N | | | | |
| 40 weeks after | 16 | 23 | N | | | | |
| 44 weeks after | 24, 25 | 15 | 2.24*, N, N | | | | |
| 48 weeks after | 20 | 19 | N | | | N | |
| 51 weeks after | 28, 31 | 22 | N | | | | N |

*borderline (+)

As shown in Tables 2 to 4, the HBV infection was observed in the chimpanzee 1 as a control, while the viral infection was not observed in the chimpanzees 2 and 3 administered with the inventive antibody together with the HBV. These results show that the inventive antibody has an excellent HBV neutralizing ability. Further, no abnormal values were found in the liver function, blood and urines tests, showing that the antibody is safe in vivo.

### Example 2

### Evaluation for HBV Neutralizing Ability in Mouse Model

### 2-1) Construction of a Plasmid Containing HBV DNA

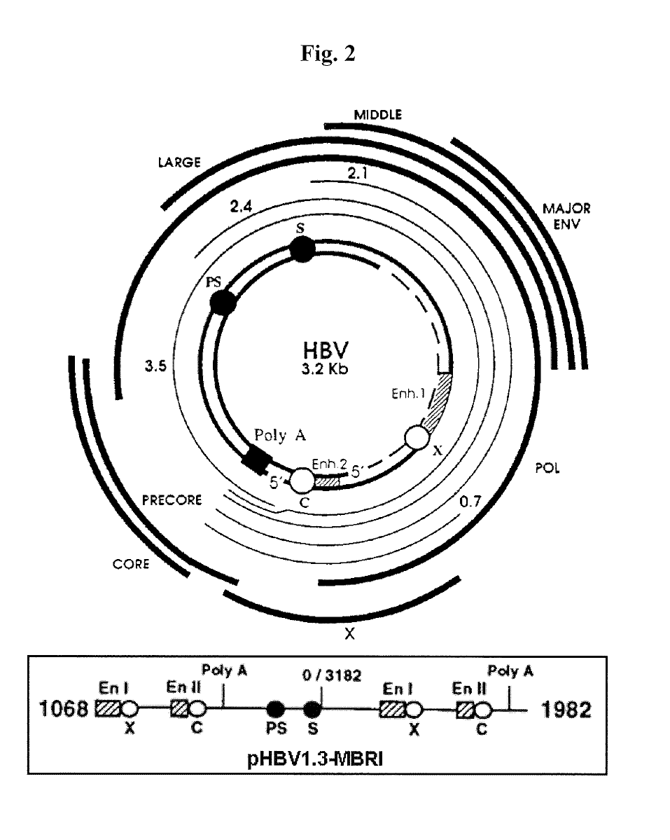

1.3 sequence of an HBV (adr subtype) gene (Gene Bank accession No. DQ683578) (HBV gene from upstream of enhancer I of an HBV genome to downstream of a polyadenylation region; see FIG. 2) was inserted into the Pmel restriction site of pcDNA3.1 (Invitrogen, USA) and the resulting plasmid pHBV1.3-MBRI was prepared by using a EndoFree Plasmid Kit (Qiagen, Germany).

### 2-2) Plasmid Injection

20 μg of the pHBV1.3-MBRI plasmid prepared in 2-1) was dissolved in a physiological saline solution to a volume corresponding to 9% of mouse weight, and injected into a tail vein of an immunodeficient C57BL/6J-Prkdcscid/SzJ female mouse (8 weeks old, Jackson laboratory, USA) at the rate of 0.3 ml/sec (hydrodynamic injection).

### 2-3) Administration of the Inventive Antibody and HBV Replication Inhibitor

Each time the HBsAg level reaches the maximum level, the inventive antibody was injected to the mouse tail vein in a concentration of 5 mg/kg based on 20 g of the mouse body weight. Only 100 μl of PBS was injected to the tail vein of the control mouse at the early stage, and after 80 days, the inventive antibody was injected as descried above. As a comparative group, 1 tablet of Zeffix (Lamivudine, GlaxoSmithKline PLC), an HBV therapeutic drug, was diluted in a distilled water, and 100 μl of the mixture was orally administered daily in a concentration of 5 mg/kg for 100 days, 50 mg/kg from the 101^{st} day and 500 mg/kg from the 124^{th} day to the 160^{th} day based on 20 g of the mouse body weight.

### 2-4) Analysis of the Change in HBsAg Level

During the experiment, eye-bleeding was performed every 3 to 4 days, and the HBsAg level and residual antibody amount in blood were analyzed by using a GENEDIA HBsAg ELISA 3.0 kit (Greencross MS, Korea).

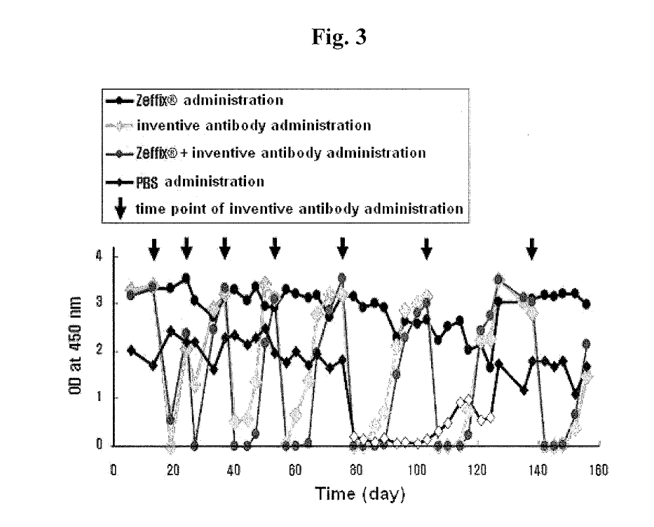

Blood samples were collected and diluted. The ELISA absorbances were measured, and absorbances measured in the 10-fold diluted blood samples were shown in FIG. 3.

As shown in FIG. 3, the HBsAg level was constantly maintained in the Zeffix-treated group, while a cycle of the decrease and increase of the HBsAg level was repeated in the inventive antibody-treated group. This result shows that the inventive antibody has an excellent HBsAg neutralizing efficacy. It is believed that the effect of Zeffix was not observed because this type of animal model might not be appropriate for the polymerase inhibitors. Further, the inventive antibody- and Zeffix-cotreated group showed a similar absorbance pattern to the inventive antibody-treated group. In the PBS-treated group, the HBsAg level was consistently maintained, but was decreased after injecting the inventive antibody at the 80^{th} day, which also shows that the inventive antibody has an excellent HBsAg neutralizing efficacy.

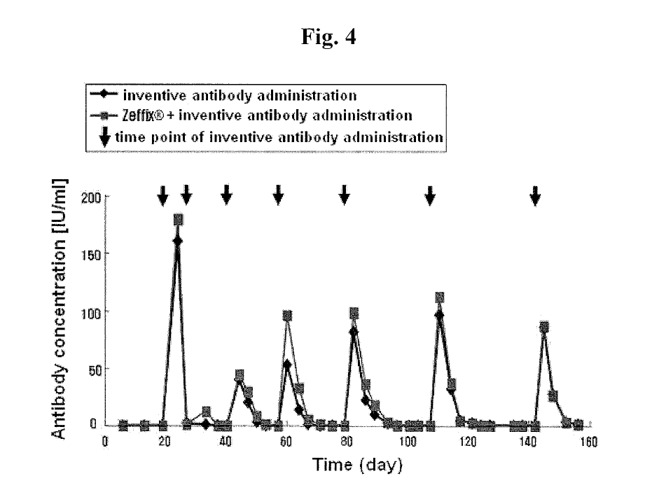

Meanwhile, the amount of the antibody in blood was measured using a standard ELISA method. Specifically, a purified recombinant HBsAg solution was diluted in PBS to a concentration of 5 μg/ml, and a 100 μl aliquot of the resultant solution was loaded to each well of a Nunc immuno module and incubated at 2 to 8° C. overnight, and the solution was removed. Then, 300 μl of a 1% BSA/PBS blocking buffer was added to each well and incubated at room temperature for 1 hour. When the reaction was completed, the blocking buffer was removed, and each 100 μl of a standard solution, test solution and spiked test solution was added thereto and incubated at room temperature for 90 min. Then, the reacting solution was removed and washed five times with a wash solution (PBS/0.05% Tween 20). And, 100 μl of a goat anti-human IgG Fab-specific peroxidase conjugate (Sigma) diluted 25,000-fold with a 1% BSA/PBS solution was added to each well and incubated at room temperature for 60 min. The plate was washed five times with the above wash solution, and 100 μl of a substrate solution (1:1 mixture of TMB microwell peroxidase substrate solutions A and B, KPL, USA) was added to each well and incubated at room temperature for 30 min. Then, 100 μl of a stopping solution (1N H₂SO₄) was added to each well, and the absorbance was measured at a measurement wavelength of 450 nm and a reference wavelength of 620˜650 nm to determine the amount of the antibody in the blood sample. The results are shown in FIG. 4.

As shown in FIG. 4, both of the inventive antibody-treated group and the antibody- and Zeffix-cotreated group exhibited similar patterns in the antibody concentration. Referring to FIG. 4, together with FIG. 3, a cycle of the increase and decrease of the antibody level (decrease and increase of the HBsAg level) by reaction of the antibody with HBsAg is repeated.

### Example 3

### Immunoprecipitation Analysis for HBV Binding Ability of the Antibody

Immunoprecipitation was carried out to examine whether the inventive antibody binds to HBV in the blood of a hepatitis B patient (provided by Ajou University, School of Medicine, Korea).

### 3-1) Preparation of Hepatitis B Patient's Blood Samples

1,000 μl of a hepatitis B patient's blood sample which was 10-fold diluted with a 0.2% BSA/PBS buffer solution was incubated with a goat anti-human IgG (Fc specific)-agarose conjugate (Research Diagnostics Inc., Flanders, N.J.) to remove an immunoglobulin.

3-2) Binding Reaction of Inventive Antibody with Goat Anti-Human IgG-Agarose Conjugate

10 μl of the inventive antibody (0.1, 0.5, 1 and 5 μg) and PBS were mixed with 50 μl of goat anti-human IgG-agarose conjugate (Research Diagnostics) and incubated at room temperature for 1 hour while stirring. 10 mg of a human immunoglobulin (I.V.-Globulin-S, Green Cross, Korea) was added thereto, and the resultant mixture was incubated at room temperature for 1 hour while stirring to block a binding region of the goat anti-human IgG-agarose conjugate. As comparative groups, each 1 μg of a blood-derived HBV antibody (Hepabig, Green Cross, Korea), TT-F9 (anti-tetanus toxoid human antibody, Green Cross, Korea), and HuS 10 (anti-hepatitis B virus surface antigen humanized antibody, Green Cross, Korea) was treated as described above.

### 3-3) Preparation of a Mixture of Antibody-Bound Goat Anti-Human IgG-Agarose Conjugate and Patient's Blood Sample

200 μl of the blood sample prepared in 3-1) was mixed with the antibody-bound goat anti-human IgG-agarose conjugate prepared in 3-2), and the resultant mixture was stirred at room temperature for 1 hour so that the antibody was subjected to a reaction with HBV in the patient's blood sample.

### 3-4) Analysis for Precipitation of HBV

The resultant solution of 3-3) was centrifuged and the supernatant was subjected to a Cobas Amplicor HBV Monitor Test, v2.0 (Roche Diagnostics, Basel, Switzerland) to measure the HBV DNA.

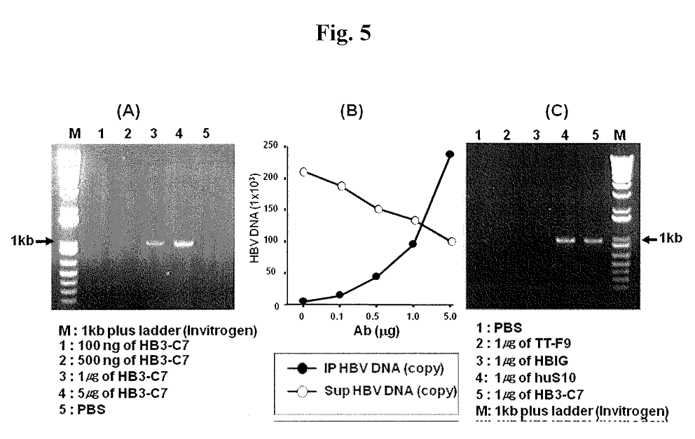

The residual agarose was washed 10 times with a 0.2% BSA/PBS buffer solution, and resuspended with 100 μl of the same buffer solution, then 5 μl of 10% SDS, 2 μl of 50 mM EDTA and 200 μg of protenase K (Sigma-Aldrich) were added and incubated at 55° C. for 30 min. Then, the supernatant was subjected to a QIAquick PCR purification kit (Qiagen, Hilden, Germany) to isolate DNA, and PCR was performed using a LiquiMix GM PCR premix (Neurotics, Korea), primers M3 (SEQ ID NO: 3) and POL8 (SEQ ID NO: 4) to amplify the HBV-specific DNA, under the following condition: denaturation for 5 min at 55° C.; 35 cycles (1 min at 95° C., 1 min at 55° C. and 1 min at 72° C.); and final extension for 10 min at 72° C. The amplified DNA was analyzed on a 1.0% agarose gel. The results are shown in FIG. 5. In (a) and (c) of FIG. 5, the analyzed samples of each lane are as follows:

FIG. 5(a)
1: group treated with 0.1 μg of the inventive antibody,
2: group treated with 0.5 μg of the inventive antibody,
3: group treated with 1 μg of the inventive antibody,
4: group treated with 5 μg of the inventive antibody,
5: PBS
   FIG. 5(c)
1: PBS,
2: group treated with 1 μg of the TT-F9 (anti-tetanus toxoid human antibody),
3: group treated with 1 μg of the Hepabig,
4: group treated with 1 μg of the HuS 10 (anti-hepatitis B virus surface antigen humanized antibody),
5: group treated with 1 μg of the inventive antibody

As shown in (a) and (b) of FIG. 5, the precipitated HBV amount was proportional to the amount of antibody used for immunoprecipitation, and the HBV amount in a supernatant after the immunoprecipitation was inversely proportional to the amount of antibody used for immunoprecipitation. Further, when the same amount of the antibodies were used, the blood-derived HBV antibody (Hepabig) did not precipitate HBV due to its weak HBV binding ability, while the inventive antibody having the strong binding ability specifically precipitated HBV (see FIG. 5(c)).

### Example 4

### Immunohistochemistry Analysis for HBV Binding Ability of the Antibody

Immunohistochemistry staining was carried out to examine whether the inventive antibody binds to an HBV-infected tissue.

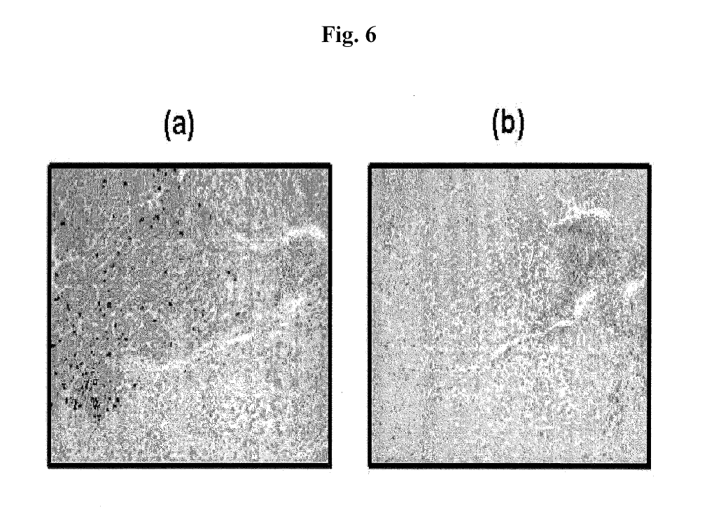

Frozen slides of the HBV-infected human liver tissues (Spring Bioscience, Fremont, Calif., USA. Catalog No. STS-025) were fixed with acetone and treated with hydrogen peroxide diluted with methanol. Then, the slides were treated with a normal rabbit serum, avidin, and biotin, sequentially. The inventive antibody and the isotype negative control antibody (human IgG 1, Sigma-Aldrich) conjugated to biotin using an Immunoprobe biotinylation kit (Sigma-Aldrich), and then StreptABComplex/HRP (Dako, Netherlands) was treated thereto. The above reagents were stained with a 3,3′-diaminobenzidine tetrahydrochloride (DAB) and counterstained with haematoxylin. The results are shown in FIG. 6.

As shown in FIG. 6, the isotype negative control antibody did not bind to the HBV-infected human liver tissue (see (b)), while the inventive antibody strongly bound thereto (see (a)).

While the invention has been described with respect to the above specific embodiments, it should be recognized that various modifications and changes may be made and also fall within the scope of the invention as defined by the claims that follow.

What is claimed is:

1. A use of a human antibody comprising a heavy chain variable region (V_{H}) having an amino acid sequence of SEQ ID NO: 1 and a light chain variable region (V_{L}) having an amino acid sequence of SEQ ID NO: 2 for the prevention or treatment of hepatitis B virus (HBV) infection or a disease caused thereby in a mammal.

2. The use of claim 1, wherein the antibody is produced from cell line HBAb-49 (KCLRF-BP-00054).

3. The use of claim 1, wherein the antibody neutralizes HBV.

4. The use of claim 1, wherein the antibody is used together with an antiviral drug.

5. The use of claim 4, wherein the antiviral drug is selected from the group consisting of interferon, an anti-HBV monoclonal antibody, an anti-HBV polyclonal antibody, a nucleoside analogue, a DNA polymerase inhibitor, an siRNA drug, and a therapeutic vaccine.

6. The use of claim 1, wherein the antibody is administered to the mammal in an amount ranging from 0.001 to 10 mg/kg (body weight)/dose.

7. A method of preventing or treating HBV infection or a disease caused thereby in a mammal, the method comprising administering thereto a human antibody comprising a heavy chain variable region (V_{H}) having an amino acid sequence of SEQ ID NO: 1 and a light chain variable region (V_{L}) having an amino acid sequence of SEQ ID NO: 2 in an effective amount.
